(19) United States
(12) Patent Application Publication
(10) Pub. No.: US 2017/0248272 A1
(43) Pub. Date: Aug. 31, 2017

(54) Safety Garment

(71) Applicant: Robert Bosch GmbH, Stuttgart (DE)

(72) Inventors: Andre Ullrich, Leinfelden-Echterdingen (DE); Joachim Schadow, Stuttgart (DE); Manfred Lutz, Filderstadt (DE); Thorsten Kuehn, Stuttgart (DE); Cornelius Boeck, Kirchheim (DE); Florian Esenwein, Leinfelden-Echterdingen (DE); Joern Stock, Wernau (DE); Joerg Maute, Sindelfingen (DE); Daniel Barth, Leinfelden-Echterdingen (DE); Juergen Wiker, Stuttgart (DE)

(21) Appl. No.: 15/519,681

(22) PCT Filed: Aug. 4, 2015
(86) PCT No.: PCT/EP2015/067939

§ 371 Date: Apr. 17, 2017

(30) Foreign Application Priority Data
Oct. 20, 2014 (DE) 10 2014 221 228.6
Apr. 14, 2015 (DE) 10 2015 206 608.8

(51) Int. Cl. F16P 3/14; A41D 1/04; A41D 3/00; A41D 1/06; A41D 19/015; A43B 3/00; A43B 7/32; A41D 13/01; A41D 13/008; A61F 9/02; A61F 9/06; A61B 5/00; G06Q 10/06; G07C 9/00; H02J 7/00; A41D 1/00

(52) U.S. Cl. CPC F16P 3/14; A41D 1/002; A41D 1/04; A41D 3/00; A41D 1/06; A41D 19/01594; A41D 19/0157; A43B 3/001; A43B 7/32; A41D 13/01; A41D 13/008; A61F 9/029; A61F 9/06; A61B 5/6804; A61B 5/0022; A61B 5/7475; G06Q 10/063114; G07C 9/00007; H02J 7/0013; A41D 2600/202; G06Q 50/265

(57) ABSTRACT

A safety garment includes at least one sensor unit for detecting at least one user-specific characteristic value and/or at least one environment-specific characteristic value. The safety garment further includes at least one communication unit configured to transmit the detected user-specific characteristic value and/or the detected environment-specific characteristic value to at least one other safety garment in order to communicate with the other safety garment.

### PRIOR ART

There are already known safety garments that have a sensor unit for sensing at least one operator-specific characteristic quantity and/or at least one environment-specific characteristic quantity.

### DISCLOSURE OF THE INVENTION

The invention is based on a safety garment having at least one sensor unit for sensing at least one operator-specific characteristic quantity and/or at least one environment-specific characteristic quantity.

It is proposed that the safety garment comprise at least one communication unit, by means of which the sensed operator-specific characteristic quantity and/or the sensed environment-specific characteristic quantity can be transmitted to at least one further safety garment for the purpose of communicating with the further safety garment. It is conceivable in this case for the communication unit to be designed to transmit the sensed operator-specific characteristic quantity and/or the sensed environment-specific characteristic quantity to at least one further safety garment that is worn, in addition to the safety garment, by a wearer of the safety garment, or to a further safety garment that is present in the vicinity of the safety garment and that is worn, for example, by another wearer. Preferably, the sensor unit and/or the communication unit are/is realized as a removable module. Thus, advantageously, the sensor unit and/or the communication unit can be replaced or, advantageously, a safety garment previously without a sensor unit and/or communication unit can be retrofitted. Moreover, it is conceivable that the module can be operated separately from the safety garment. Thus, advantageously, a great variety of objects can be equipped with the module. It is also conceivable in this case for the module to comprise, in addition to the sensor unit and/or the communication unit, other units, considered appropriate by persons skilled in the art, such as, for example, an output unit, an input unit, an evaluation unit, a projection unit, a lighting unit, or the like.

An “operator-specific characteristic quantity” is to be understood here to mean, in particular, a characteristic quantity that defines at least one vital value of a wearer of the safety garment, and that acts on a wearer of the safety garment and/or that is dependent on a behavior of a wearer of the safety garment, such as, for example, a direction and/or a location of an application of force by a wearer of the safety garment upon the safety garment. The operator-specific characteristic quantity in this case may be realized as a type of operator-specific safety-garment burden, as a wearer burden such as, for example, a noise burden and/or a vibration burden, as a pulse of a wearer of the safety garment, as a body temperature of a wearer of the safety garment, as a fatigue characteristic quantity of a wearer of the safety garment, as a wearer orientation characteristic quantity, as a movement characteristic quantity, or as another operator-specific characteristic quantity considered appropriate by persons skilled in the art. An “environment-specific characteristic quantity” is to be understood here to mean, in particular, a characteristic quantity that defines an environment surrounding the safety garment. The environment-specific characteristic quantity in this case may be realized as an ambient pressure, as an ambient temperature, as an ambient sound level, as an ambient gas characteristic quantity, as an ambient potential-difference characteristic quantity, as an ambient humidity characteristic quantity, as an ambient acid and/or ambient base characteristic quantity, or as another environment-specific characteristic quantity considered appropriate by persons skilled in the art.

The term “safety garment” is intended here to define, in particular, a garment that has at least one passive protective unit and that, in particular, is specially designed to be worn by a wearer during a work operation. A “passive protective unit” is to be understood here to mean, in particular, a protective unit designed to passively protect a wearer, at least against mechanical and/or electrical loads. The safety garment may thus be realized as a work jacket, as a work vest, in particular as a safety vest, as work trousers, as a work shoe, as a safety helmet, as safety glasses, as a work glove, as a work belt, or the like. Preferably, the safety garment comprises at least one reflector unit for reflecting incident light beams. The sensor unit of the safety garment additionally comprises at least one position sensor element, which is designed to sense at least one position characteristic quantity, in particular at least one global position characteristic quantity and/or at least one relative work-region position characteristic quantity. For this purpose, the sensor unit preferably comprises at least one position sensor element realized as a GPS sensor element, by means of which a global position of the safety garment can be sensed. It is also conceivable, however, that the sensor unit has a different position sensor element, considered appropriate by persons skilled in the art, for sensing a position characteristic quantity realized as a global position, such as, for example, a compass position sensor element, a Galileo position sensor element, a GLONASS position sensor element, a Beidou position sensor element, or the like. Moreover, the sensor unit preferably has at least one sensor element realized as a work-region position sensor element, which is designed, for example by means of a propagation time measurement via a WLAN network or via a mobile telephony network, to enable a relative position of the safety garment to be identified within a work area, such as, for example, an iBeacon locating element. It is thus possible, advantageously, to achieve sensing of a position of the safety garment that, for example in the case of an emergency situation, enables the location of the safety garment, and thus of the wearer of the safety garment, to be determined in a reliable manner. It is conceivable in this case that sensing of at least one position characteristic quantity is effected only when a hazard situation is identified. It is thus conceivable that sensing of at least one position characteristic quantity is deactivated during absence of a hazard situation.

The communication unit is preferably realized as a wireless communication unit. The communication unit in this case may be realized as a WLAN communication unit, as a Bluetooth communication unit, as a radio communication unit, as an RFID communication unit, as an NFC unit, as an infrared communication unit, as a mobile telephony communication unit, as a Zigbee communication unit, or the like. Particularly preferably, the communication unit is designed for bidirectional data transmission. In an alternative design, the communication unit is realized as a wired communication unit such as, for example, a LAN communication unit, a USB communication unit, as a Powerline communication unit, as a CAN-bus Communication unit, as an Ethernet communication unit, as a twisted-pair cable communication unit (CAT5 or CAT6), or the like. It is also conceivable, however, that the communication unit, as an alternative to being designed for wireless or wired communication, is designed for wireless and wired communication. The term “transmit” is intended here to define, in particular, a sending and/or receiving of data, in particular electronic data.

The design of the safety garment according to the invention makes it possible, advantageously, for differing safety garments to communicate with each other, in order thus to achieve, particularly advantageously, a high degree of work convenience and/or wear convenience. For example, a high degree of wearing convenience can be achieved, advantageously, in that at least one convenience function is monitored on the basis of sensor data or on the basis of a communication of the safety garment and of the further safety garment, and is adapted according to the sensor data or according to the communication. Moreover, advantageously, hazard situations can be identified as a result of a communication between the individual safety garments, and a wearer of the safety garment/safety garments can advantageously be warned and/or protected against hazards. It is thus possible, particularly advantageously, to provide a safety garment that can provide a high degree of wearer safety and/or wearer convenience.

Furthermore, it is proposed that the communication unit be designed to communicate with at least one external unit realized so as to be different from a safety garment, for the purpose of exchanging electronic data. Thus, also conceivable is a safety garment having at least one sensor unit for sensing at least one operator-specific characteristic quantity and/or at least one environment-specific characteristic quantity, and having at least one communication unit designed to communicate with at least one external unit, in particular with at least one external unit realized so as to be different from a safety garment, for the purpose of exchanging electronic data. The external unit may be realized as a smartphone, as a personal computer, as a laptop, as a netbook, as a tablet, as a company central computer, as a hand-held power tool, as an output unit such as, for example, a loudspeaker, or as another external unit considered appropriate by persons skilled in the art. In the case of a design as a smartphone, as a personal computer, as a laptop, as a netbook or as a tablet, an app is preferably provided for communicating with the communication unit. It is also conceivable, however, that the external unit is realized as an external, transportable operating unit, as a fixedly installed operating unit at a work-station of an operator, as a synchronization unit of a place of use, which is fixedly installed in a space and can be controlled by a control center, such as, for example, as a result of company regulations/safety specifications, as a body characteristic quantity monitoring unit, or as another central or decentralized operating unit, input station and/or central or decentralized terminal, considered appropriate by persons skilled in the art. Thus, advantageously, synchronization of electronic data can be achieved. If, for example, the presence of a wearer of the safety garment, in particular the wearing of the safety garment, and the presence and/or existence of the external unit is identified by means of a sensor element of the sensor unit, a connection is established, at least partially automatically, between the communication unit and the external unit. Settings and/or data stored in the external unit can thus preferably be transmitted directly to the safety garment and/or from the safety garment to the external unit. These may be individual settings of a wearer, or company regulations. In addition, the communication unit can transmit, for example, a noise burden on a wearer of the safety garment, for the purpose of monitoring compliance with a burden limit, and/or a possible payment of allowances to a company control center, or the like.

Preferably, a communication between the safety garment and the external unit, in particular an external unit realized as a synchronization unit of a place of use, which is fixedly installed in a space and can be controlled by a control center, can be used to include also a work area. The work area consists, in particular, of buildings, spaces, rooms, construction sites, or the like. This work area in this case can preferably be managed by a facility management (FCM) and/or monitored by a company control center. In this case, particularly in work areas used for commercial purposes, rules and authorizations, such as, for example, rules relating to working safety, can be established. Thus, the work area can be sub-divided according to a degree of hazard, with corresponding rules and authorizations being assignable to individual sub-regions of the work area, such as, for example, the wearing of appropriate protective equipment, the specification of access authorization, the existence of more extensive training/instruction for compliance with a working safety regulation, etc. Advantageously in this case, the design according to the invention makes it possible to achieve automatic verification of these rules and authorizations. Thus, advantageously, hazard regions in a work area can be identified, and access authorizations, or the execution of particular works or of works performed by means of particular tools and/or power tools, can be assigned in dependence on the safety garment worn. Moreover, it is conceivable that a smartphone is provided as a “router” and/or as an exchange between the communication unit of the safety garment and differing network technologies of external units. Use of a dedicated company smartphone is very advantageous in this case. Usually, a smartphone has commonly used short-range radio relay standards such as, for example, Bluetooth and NFC, and long-range radio relay standards such as, for example, WLAN, UMTS, GSM, LTE, or the like. The design according to the invention can therefore be used, advantageously, to effect convenient, in particular central, setting of characteristic quantities. In addition, advantageously, single-person monitoring can be achieved, such that a company control center can be informed, advantageously, about a state of a wearer of the safety garment, and in particular about any hazard situations. Thus, advantageously, a high degree of safety can be ensured for a wearer of the safety garment.

In addition, advantageously, so-called work reports, for example on servicing works, can be compiled and recorded automatically. It is thereby possible, advantageously, to document at what location work was performed, by whom, when, for how long and with what safety garment. If, for example, a case of damage or an accident occurs, an automatically compiled report can advantageously provide, for example, facts for proof of compliance with a duty of care. By means of processing of sensed position characteristic quantities it is possible, advantageously, to realize a precise representation of a work location that, by means of the communication unit, can be transmitted, for example, to an external unit of a control center. It is thus made possible, advantageously, for example in the control center, to have an overview of locations of various wearers of safety garments according to the invention. It is also conceivable, however, that differing safety garments communicate by means of the respective communication unit, in order to exchange individual position characteristic quantities. Mutual determination of position can thus be effected between differing wearers. This makes it possible, for example, for information signifying individual locations of differing wearers to be transmitted between the wearers. Moreover, advantageously, it is made possible to find lost items that can communicate with the communication unit.

It is additionally proposed that the safety garment comprise at least one output unit, which is designed to output items of information in dependence on the sensed operator-specific characteristic quantity, the sensed environment-specific characteristic quantity and/or in dependence on data received by means of the communication unit. The output unit in this case may be realized as a haptic, acoustic and/or optical output unit. Preferably, the output unit comprises at least one output element, which is disposed on the safety garment and designed to output at least one item of information. In this case, if the safety garment is realized as a safety helmet, the output element may be integrated as a display into a visor of the safety helmet. Moreover, if the safety garment is realized as safety glasses, the output element may be integrated as a display into a lens of the safety glasses. Further, if the safety garment is designed as a welding helmet, the output element may be integrated as a display into a viewing window of the welding helmet. Furthermore, if the safety garment is designed as a safety glove, as a work jacket, as a work vest, in particular a work vest, in particular as a safety vest, as work trousers, as a work shoe or the like, the output element may be integrated as a display into an outer surface of the safety garment. The output unit can preferably be controlled by open-loop and/or closed-loop control, by means of an open-loop and/or closed-loop control unit. The design according to the invention advantageously enables information to be output to a wearer of the safety garment. Thus, advantageously, a wearer of the safety garment can be protected against a hazard situation. Moreover, advantageously, other persons present in the area around the wearer of the safety garment can likewise be warned, advantageously, against a hazard situation.

It is additionally proposed that at least work instructions that are dependent, at least, on data received by means of the communication unit can be output by means of the output unit. Preferably, work instructions that are dependent, at least, on characteristic quantities sensed by means of the sensor unit can be output by means of the output unit. It is thus possible, advantageously, to output work instructions that can be matched to an individual manner of working of a wearer of the safety garment.

Furthermore, it is proposed that the safety garment comprise at least one evaluation unit that is designed to evaluate a safety garment combination, at least in dependence on a communication with the further safety garment. It can thus advantageously be identified, at least, whether a wearer of the safety garment is wearing a further safety garment that is suitable for their work area or their work task. The evaluation unit is designed, at least, in the case of evaluation of the safety garment combination, to evaluate data, of the further safety garment and/or of the sensor unit of the safety garment, preferably transmitted by means of the communication unit. For example, the presence of a further safety garment can be sensed by means of the communication unit and/or by means of the sensor unit. By means of the communication unit, for example, this safety garment combination can be transmitted to a control center. It is thereby possible, advantageously, to deduce a correctly worn protective equipment. Moreover, it is conceivable that a position characteristic quantity of the safety garment and/or of the further safety garment is transmitted to the control center. If, for example, following a break, a wearer forgets to put on the further safety garment and continues with their work, a position of the further safety garment that has not been put on can be registered, for example, in the control center, and a corresponding warning can be output to the wearer by means of the output unit.

It is additionally proposed that the safety garment comprise at least one evaluation unit that is designed to evaluate a sensed environment, at least in dependence on an environment-sensing sensor element, in particular a camera, of the sensor unit, in order to make possible a work instruction. As an alternative or in addition to the environment-sensing sensor element, which is preferably realized as a camera, the sensor unit may comprise an environment-sensing sensor element realized in a different manner, or further environment-sensing sensor elements, which is/are realized, for example, as a temperature sensor element/temperature sensor elements, as a material characteristic quantity sensor element/material characteristic quantity sensor elements, as a noise sensor element/noise sensor elements, or the like. The evaluation unit may evaluate a workpiece, or a base on which the workpiece is deposed, for example on the basis of a characteristic quantity sensed by means of the environment-sensing sensor element, in order to assess, for example, a possibility of projecting an instruction onto a surface of the workpiece or base. The evaluation unit in this case may evaluate, for example, a contrast of the workpiece or of the base, a brightness of the environment, or the like. Thus, advantageously, an output of a work instruction can be adapted to an environment. Further, it is conceivable that a workpiece can be sensed by means of the environment-sensing sensor element, and a location at which work is performed on the workpiece can be evaluated on the basis of an evaluation by means of the evaluation unit, a work instruction being able to be displayed directly on the workpiece, or work instructions that are dependent on the sensed workpiece and/or on the evaluation by the evaluation unit being able to be output by means of the output unit. If, for example, it is intended to make a chamfer at a particular location on a workpiece, or if it is intended, for example, to remove a weld seam, the location for making the chamfer, or the weld seam, can be sensed, for example, by means of the environment-sensing sensor element acting in combination with the evaluation unit, and a corresponding work instruction can be output. It is conceivable in this case that, for example, the sensed tool can be represented in the output unit, and the location for making the chamfer, or the weld seam, can be indicated by means of a visual symbol in a representation of the tool, the output unit in this case preferably being realized so as to be at least substantially integral with a lens of safety glasses or with a visor of a safety helmet. It can be ensured, advantageously, that a work instruction can be safely seen by a wearer of the safety garment. In a further application of the evaluation unit according to the invention, it is conceivable that application parameters are stored in a storage unit of the safety garment. If, for example, a workpiece temperature determined by means of the sensor unit is higher than a temperature limit value stored in the storage unit, this can be output, for example, by means of the output unit, or control commands such as, for example, a safety switch-off command, or the like, can be transmitted by means of the communication unit to an external unit that is realized as a hand-held power tool.

It is additionally proposed that the safety garment comprise at least one input unit, at least for inputting operator-specific control commands, wherein, for the purpose of controlling an external unit, in particular a hand-held power tool, the operator-specific control commands can be transmitted to the external unit by means of the communication unit. The input unit in this case may be realized as an optical, acoustic or haptic input unit. The input unit preferably has at least one input element such as, for example, a key, a touch-sensitive display, a rocker switch, or the like, which is disposed on an outer surface of the safety garment that is easily accessed by a wearer of the safety garment. By means of the design according to the invention, it is possible, advantageously, to achieve extended operating functions of the external unit.

Furthermore, it is proposed that the input unit have at least one gesture control function and/or voice control function. The input unit thus preferably comprises at least one gesture sensing element, in particular a camera, and/or a voice sensing element, in particular a microphone. The design according to the invention advantageously makes it possible to achieve convenient operation of the external unit and/or convenient inputting of setting parameters for individual setting of the safety garment.

It is additionally proposed that the safety garment comprise at least one actuator unit, which can be controlled at least in dependence on the sensed operator-specific characteristic quantity, in dependence on the sensed environment-specific characteristic quantity and/or in dependence on data received by means of the communication unit. The actuator unit in this case may be designed to enable, in particular to set, convenience functions and/or safety functions. For example, it is conceivable that a fit of the safety garment can be adapted, by means of the actuator unit, in dependence on the sensed operator-specific characteristic quantity, in dependence on the sensed environment-specific characteristic quantity and/or in dependence on data received by means of the communication unit. Likewise conceivable, furthermore, are convenience functions and/or safety functions of the safety garment, considered appropriate by persons skilled in the art, that can be influenced by means of the actuator unit. The design according to the invention makes it possible, advantageously, to realize a high degree of wear convenience and/or a high standard of safety.

It is additionally proposed that the actuator unit be designed, at least, to act actively on a wearer as a result of identification of a hazard situation. It is conceivable, for example, that the sensor unit of the safety garment, in particular of a work shoe, comprises at least one potential-difference sensor element designed to sense an electric potential between a wearer of the safety garment and, for example, a floor on which the wearer of the safety garment is present. The actuator unit preferably has at least one haptic actuator element, which is designed, by generating vibrations, to make a wearer aware, for example, of the presence of a high electric potential. It is conceivable in this case that information regarding the sensed electric potential can additionally be output by means of the output unit, in order to inform a wearer, or persons in an area around the wearer, of the presence of an electric potential. If there is a flow of voltage though the wearer, it is conceivable that a warning signal can be output by means of the output unit. Advantageously, persons present in the vicinity can thereby be informed of the hazard situation. Moreover, it is conceivable that an emergency signal can be emitted, at least partially automatically, in particular if a voltage limit value is exceeded, or as a result of a critical vital value of a wearer being sensed by means of the sensor unit. Furthermore, it is conceivable that the actuator unit has at least one actuator element, in particular at least one smart-material actuator element, which is designed, in the case of persistence of the flow of voltage through the wearer, to force a movement, for example on limbs of the wearer, in order, for example, to move, in particular to bend, a leg or an arm in an abrupt manner, in order thereby to interrupt a through-flow of voltage, in particular as a result of moving away from a voltage source. By means of the design according to the invention, advantageously, a high degree of wearer safety can be ensured.

Furthermore, it is proposed that the safety garment comprise at least one lighting unit for illuminating a work area, wherein the lighting unit can be controlled at least in dependence on the sensed operator-specific characteristic quantity, in dependence on the sensed environment-specific characteristic quantity and/or in dependence on data received by means of the communication unit. Particularly preferably, the lighting unit is realized as an LED lighting unit. It is also conceivable, however, that the light unit is of a different design, considered appropriate by persons skilled in the art, such as, for example, designed as a noble-gas lighting unit, as a laser-light lighting unit, or the like. The design according to the invention makes it possible, advantageously, to work safely in dark or poorly illuminated work areas. Thus, advantageously, a good view of a work area can be achieved.

It is additionally proposed that the safety garment comprise at least one projection unit, which is designed to project at least one item of information onto a base, wherein the projection unit can be controlled at least in dependence on the sensed operator-specific characteristic quantity, in dependence on the sensed environment-specific characteristic quantity and/or in dependence on data received by means of the communication unit. The projection unit is preferably designed to project at least one work instruction onto a surface of an object such as, for example, a workpiece, in dependence on the sensed operator-specific characteristic quantity, in dependence on the sensed environment-specific characteristic quantity and/or in dependence on data received by means of the communication unit. The projection unit in this case may be realized as an LED projection unit, as a laser projection unit, or as another projection, considered appropriate by persons skilled in the art. By means of the design according to the invention, advantageously, items of information are rendered legible in a reliable manner. Moreover, a large surface area can be used for displaying items of information.

It is additionally proposed that the sensor unit have at least one eye sensor element, which is designed to sense at least one eye characteristic quantity of a wearer, in particular of a wearer of a safety garment, wherein the eye characteristic quantity can be transmitted to the further safety garment and/or to an external unit by means of the communication unit. Preferably, the eye sensor element is designed, at least, to sense an eye characteristic quantity realized as a state (open, closed, blink rate, or the like) of at least one eye of a wearer, or as a viewing direction of an eye of a wearer. It is also conceivable, however, that the eye sensor element is designed, additionally or alternatively, to sense different or additional eye characteristic quantities, considered appropriate by persons skilled in the art. Preferably, the eye characteristic quantity sensed by means of the eye sensor element can be evaluated by means of the evaluation unit. It is thus possible, advantageously, to deduce the attentiveness of a wearer of the safety garment, since it is possible to identify, for example, that the wearer has directed their view away from a workpiece on which work is to be performed and/or from a hand-held power tool. It is conceivable that, for example, a safety switch-off of the external unit, in particular of the hand-held power tool, can be initiated in dependence on the sensed eye characteristic quantity and a hazard potential identified as a result of an evaluation by means of the evaluation unit. Moreover, advantageously, identification of wearer fatigue can be achieved in dependence on the sensed eye characteristic quantity and as a result of an evaluation by means of the evaluation unit. Advantageously, a warning signal can be output, or a safety switch-off of the external unit can be effected as a result of transmission of electronic data by means of the communication unit.

Furthermore, it is proposed that the safety garment comprise at least one energy supply unit, which is designed to supply at least the sensor unit and the communication unit with energy in dependence on a handling of an external unit, in particular a hand-held power tool. The energy supply unit is preferably realized as a contactless energy supply unit. The energy supply unit thus preferably comprises at least one primary coil for receiving electrical energy. Preferably, the external unit, in particular the hand-held power tool, has at least one secondary coil, which is designed to transfer electrical energy to the energy supply unit of the safety garment when the safety garment is in the vicinity of the external unit. Moreover, it is conceivable that the energy supply unit has at least one storage battery for storing electrical energy. Further, it is also conceivable that the energy supply unit is designed to use renewable energy for energy supply, such as, for example, as a solar module, wind-power module, or the like. The design according to the invention advantageously enables appropriate use to be made of an energy supply of the external unit, in particular, of the hand-held power tool, for supplying energy at least to the sensor unit and the communication unit. Particularly advantageously, it is possible to do without an additional energy supply for the safety garment, in particular if the safety garment is designed such that it is separate from an additional storage battery.

Also proposed is a safety system, having at least one safety garment according to the invention, and having at least one further safety garment, and/or having at least one external unit, with which the safety garment communicates, by means of the communication unit, for the purpose of exchanging electronic data. In respect of a geometric design, the further safety garment preferably differs in design from the safety garment. In respect of a safety design, the further safety garment is preferably of a design that is at least substantially similar. The safety garment may be realized, for example, as a work jacket, and the further safety garment may be realized as work trousers, the further safety garment having at least substantially the same electrical and/or electronic units, such as, for example, a communication unit, a sensor unit, etc., as the safety garment. In respect of the design of the safety garment and of the further safety garment, any combinations of safety garments considered appropriate by persons skilled in the art are conceivable. Preferably, the safety system comprises a multiplicity of safety garments that each have at least one communication unit, for communicating with one another, and/or at least one sensor unit, for sensing at least one operator-specific characteristic quantity and/or at least one environment-specific characteristic quantity. Preferably, the safety system comprises, for example, a safety garment realized as a work jacket, a further safety garment realized as work trousers, an additional safety garment realized as a safety helmet, an additional safety garment realized as a safety glove, an additional safety garment realized as a safety shoe, and/or an additional safety garment realized as safety glasses. The design according to the invention advantageously enables a high degree of safety to be achieved for a wearer of the safety garment.

It is additionally proposed that the sensor unit of the safety garment have at least one position sensor element, wherein the external unit has at least one hazard identification function, which is designed at least to evaluate a hazard situation of a wearer of the safety garment, at least in dependence on a position characteristic quantity sensed by means of the position sensor element. For example, it is conceivable that sensor unit of the safety garment senses a position characteristic quantity that can be transmitted, by means of the communication unit, to a control center designed to monitor a work area. Thus, advantageously, a location of a wearer wearing the safety garment is known in the control center. A work-area characteristic quantity such as, for example, a rail traffic characteristic quantity, a motor-vehicle traffic characteristic quantity, or the like, can preferably be matched to the sensed position characteristic quantity, in dependence on a work area. The work-area characteristic quantity in this case can preferably be transmitted to the safety garment by means of the communication unit, and output by means of the output unit. A wearer of the safety garment can preferably be informed, via the output unit, about a current time period until arrival of a train at a work location. Moreover, it is conceivable that, as a result of a communication with the control center, a vehicle driver such as, for example, a train driver, a car driver, or the like, can also be informed about a location of a wearer wearing the safety garment. If, for example, a hazard to a wearer wearing the safety garment is registered in the control center, a warning message can be output to the wearer and/or to the vehicle driver. If, for example, no reaction of a wearer to a warning message can be sensed, a counter-measure can be initiated, preferably at least partially automatically, such as, for example, emergency braking of the train, in order for a risk of injury to a wearer of the safety garment to be limited to a low level.

It is additionally proposed that the at least one external unit have at least one access control function, which is designed to monitor, enable or block access of a wearer of the safety garment and/or of the further safety garment to a work area or a space. By means of the access control function it is possible to monitor, for example, to which space and/or region of a work area a wearer of the safety garment has access, and what work authorization the wearer has, in particular with respect to safety instructions. Thus, for example, the issue of a hand-held power tool to the wearer of the safety garment can be recorded, in that a communication is effected between the safety garment and the hand-held power tool, with data additionally being able to be transmitted to the external unit, in particular a control center. Owing to the access control function, it is possible to sense, for example, entrance into a region of a work area in which servicing work can be performed. If the sensor unit of the safety garment senses a slippery base surface, for example by means of an acceleration sensor, a safety function of the hand-held power tool such as, for example, a kick-back function in the case of an angle grinder, can be automatically activated or set by means of a communication with the hand-held power tool. Moreover, it is conceivable that the safety garment transmits to the external unit, by means of the communication unit, an item of information with regard to the presence of a slippery base surface and, on the basis thereof, for example, that a member of cleaning personnel is sent to remedy the slippery base surface. It is additionally conceivable that, for example in the case of work performed on a workpiece in which sparks are generated, that a gas sensor element of the sensor unit of the safety garment senses the presence of a highly flammable gas, whereupon, for example, the hand-held power tool can be deactivated automatically, a warning can be output to the wearer by means of the output unit, and/or the presence of the highly flammable gas in a region of the work area can be communicated to the external unit, in order to initiate appropriate counter-measures. By means of the design according to the invention, advantageously, a simple access control can be achieved.

Furthermore, it is proposed that the at least one external unit have at least one garment monitoring function, which is designed to monitor a safety garment combination that is contingent upon a work region and/or a work assignment, in dependence on safety regulations and/or operating-area regulations. It is thus possible, advantageously, to realize automatic monitoring of the obligation to wear safety garments.

It is additionally proposed that the at least one external unit be realized as a hand-held power tool or as a company control center. A “hand-held power tool” is to be understood here to mean, in particular, a power tool, for performing work on workpieces, that can be transported by an operator without the use of a transport machine. In particular, the hand-held power tool has a mass of less than 40 kg, preferably less than 10 kg, and particularly preferably less than 5 kg.

Preferably, the external unit realized as a hand-held power tool has at least one communication unit for exchanging electronic data with the communication unit of the safety garment. In particular, the external unit realized as a hand-held power tool has at least one sensor unit, for sensing characteristic quantities specific to a hand-held power tool and/or workpiece. Preferably, the characteristic quantities specific to a hand-held power tool and/or workpiece can be transmitted at least to the safety garment, in particular by means of a communication between the communication unit of the hand-held power tool and the communication unit of the safety garment. Preferably, the characteristic quantities specific to a hand-held power tool and/or workpiece can be output by means of the output unit of the safety garment, in particular for the purpose of informing an operator of the hand-held power tool. The characteristic quantity specific to a hand-held power tool and/or workpiece may, for example, a storage-battery charge-state characteristic quantity, a counter characteristic quantity (number of applications/switch-on operations, or the like), an operating-mode type characteristic quantity (for example first gear, second gear, hammer drilling, chipping, drilling, or the like), an absolute rotational speed, a percentage of a maximum rotational speed, an output power, a percentage of a maximum output power, an overload characteristic quantity, a leveling characteristic quantity (for example a deviation from a vertical orientation, from a horizontal orientation and/or from an orientation at a prescribed angle, or the like), a service characteristic quantity (for example brush change, change of transmission grease, etc.), or the like.

The design of the safety system according to the invention makes it possible, advantageously, to achieve a communication between the hand-held power tool and the safety garment in order, for example, to achieve user-friendly inputting of operating commands and/or to output to an operator items of information relating to a hand-held power tool.

The invention is additionally based on an eye protection device, in particular safety glasses or a safety visor, for use in a safety system according to the invention, having at least one communication unit, for exchanging electronic data, which is designed to communicate with at least one hand-held power tool, and having at least one output unit, which is designed to output items of information in dependence on data received by means of the communication unit.

It is proposed that the communication unit be designed, at least, to communicate with an external unit that is different from a hand-held power tool. The eye protection device is preferably realized as a safety garment. In particular, the eye protection device is realized to sense at least one operator-specific characteristic quantity and/or at least one environment-specific characteristic quantity, separately from a sensor unit. It is also conceivable, however, that the eye protection device, in an alternative design, has a sensor unit for sensing at least one operator-specific characteristic quantity and/or at least one environment-specific characteristic quantity. Moreover, it is conceivable that the eye protection device is operable independently of the safety system according to the invention.

Preferably, the communication unit of the eye protection device is realized as a wireless communication unit. It is also conceivable, however, that the communication unit of the eye protection device, alternatively or additionally, has a wired communication capability. In respect of a design of a communication unit of the eye protection device, reference may be made, in principle, to the description of the communication unit of the safety system and/or of the safety garment, which communication unit is of a design that is at least substantially similar to the that of the communication unit of the eye protection device. In respect of a design of the output unit of the eye protection device, reference may be made, in principle, to the description of the output unit of the safety system and/or of the safety garment, which output unit is of a design that is at least substantially similar to that of the output unit of the eye protection device.

Preferably, the external unit that is different from a hand-held power tool is realized as a mobile device, as a charging device, as a company control center, or the like. The mobile device may be a mobile telephone, in particular a smartphone, a laptop, a netbook, a tablet, or the like.

Advantageously, a high degree of operating convenience can be achieved by means of the design of the eye protection device according to the invention. Moreover, advantageously, it can be instrumental in the attentiveness and/or a viewing direction of an operator continuing to be directed toward a workpiece on which work is to be performed. Advantageously, a display of characteristic quantities specific to a hand-held power tool and/or workpiece can be conveniently expanded by further data.

Furthermore, it is proposed that the communication unit of the eye protection device be designed, at least, to communicate with an external unit realized as a mobile device, in order to exchange electronic data with the mobile device. In particular, the mobile device is realized as a smartphone. Advantageously, it is possible to achieve outputting of incoming short message calls such as, for example, an SMS, or the like. Moreover, advantageously, personal time schedules can be displayed by means of the output unit. Advantageously, a great variety of information can be realized by means of a single item.

It is additionally proposed that the communication unit of the eye protection device be designed, at least, to communicate with an external unit realized as a charging device, in order to receive at least one charge characteristic quantity from at least one storage battery that can be charged by means of the charging device. The charge characteristic quantity may be realized as a charge state, as a remaining charge duration, as a charge capacity, as a charging voltage, or the like. The design according to the invention advantageously enables an operator to be provided with an item of information that informs the operator about a state of a storage battery disposed in a charging device. Advantageously, a storage-battery change interval can be planned in dependence on an output of the output unit, to enable advantageous use to be made of a work time period.

It is additionally proposed that the communication unit of the eye protection unit be designed, at least, to communicate with an external unit realized as a company control center, in order to receive at least project data. The project data may be, for example, construction diagrams, parts lists, work requirements, work plans, information relating to a contact partner, information relating to a work location, or the like. By means of the design according to the invention, advantageously, an operator can be informed in good time concerning changes or new developments in project data. Advantageously, reference data can be conveniently compared with actual data. As a consequence, advantageously, errors can be avoided, or kept to a minimum.

Additionally proposed is a method for operating the safety system according to the invention. Preferably, the method has at least one method step in which the safety garment is coupled, at least partially automatically, to the further safety garment and/or to the external unit. Preferably, the method has at least one method step in which a recording function can be initiated, at least partially automatically. Advantageously, a wearer of the safety garment can be monitored at least partially automatically.

The safety garment according to the invention, the safety system according to the invention, the eye protection device according to the invention and/or the method according to the invention are/is not intended in this case to be limited to the application and embodiment described above. In particular, the safety garment according to the invention, the safety system according to the invention, the eye protection device according to the invention and/or the method according to the invention may have individual elements, components and units, and method steps, that differ in number from a number stated herein, in order to fulfill a principle of function described herein. Moreover, in the case of the value ranges specified in this disclosure, values lying within the stated limits are also to be deemed as disclosed and applicable in any manner.

### DRAWING

Further advantages are disclosed by the following description of the drawing. The drawing shows an exemplary embodiment of the invention. The drawing, the description and the claims contain numerous features in combination. Persons skilled in the art will also expediently consider the features individually and combine them to create appropriate further combinations.

There are shown in:

FIG. 1 a safety system according to the invention, having at least one safety garment, and having at least one further safety garment, in a schematic representation,

FIG. 2 a communication diagram of the safety system, having external units, in a schematic representation,

FIG. 3 a communication diagram of the safety system to enable monitoring of a spatial and/or garment combination, in a schematic representation,

FIG. 4 a detail view of an additional safety garment of the safety system according to the invention, in a schematic representation,

FIG. 5 a detail view of a further additional safety garment of the safety system according to the invention, in a schematic representation,

FIG. 6 a detail view of a further additional safety garment of the safety system according to the invention, in a schematic representation, and

FIG. 7 a schematic representation of a use of the eye protection device according to the invention in a safety system according to the invention.

### DESCRIPTION OF THE EXEMPLARY EMBODIMENT

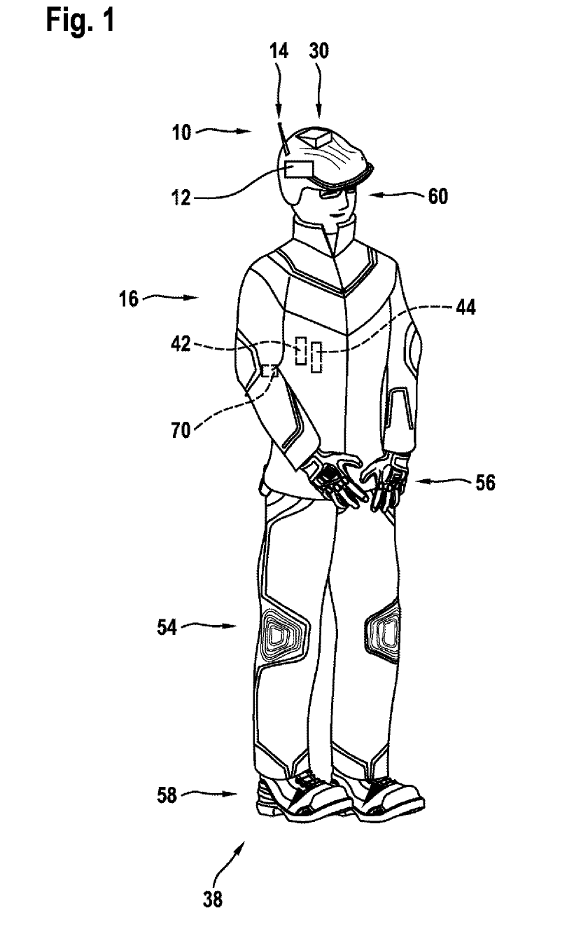

FIG. 1 shows a safety system 38, having at least one safety garment 10, and having at least one further safety garment 16, with which the safety garment 10 communicates, by means of a communication unit 14 of the safety garment 10, for the purpose of exchanging electronic data. The communication unit 14 of the safety garment 10 is preferably realized as a wireless communication unit. The safety garment 10 and/or the further safety garment 16 in this case may be realized as a work jacket, as a work vest, in particular as a safety vest, as work trousers, as a work shoe, as a safety helmet, as safety glasses, as a work glove, as a work belt, or the like. The further safety garment 16 preferably differs from the safety garment 10 in respect of a geometric design. In the case of the exemplary embodiment represented in FIG. 1, the safety garment 10 is realized, exemplarily, as a safety helmet, in particular for the purpose of explaining a functionality of the safety garment 10 and/or of the safety system 38. It is also conceivable, however, that the safety garment 10 is of a different design, considered appropriate by persons skilled in the art. In the case of the exemplary embodiment represented in FIG. 1, the further safety garment 16 is realized, exemplarily, as a work jacket, in particular for the purpose of explaining a functionality of the further safety garment 16 and/or of the safety system 38. It is also conceivable, however, that the further safety garment 16 is of a different design, considered appropriate by persons skilled in the art.

The safety garment 10 comprises at least one sensor unit 12, for sensing at least one operator-specific characteristic quantity and/or at least one environment-specific characteristic quantity. For this purpose, the sensor unit 12 comprises at least one environment-sensing sensor element 24 and/or an operator-sensing sensor element (not represented in greater detail here). The environment-sensing sensor element 24 and/or the operator-sensing sensor element in this case may be of any design considered appropriate by persons skilled in the art, such as, for example, designed as a safety-garment load-type sensor, as a wearer-burden sensor (noise burden sensor, vibration burden sensor, or the like), as a pulse sensor, as a body temperature sensor, as a fatigue sensor, as a wearer orientation sensor, as a motion sensor, as an ambient pressure sensor, as an ambient temperature sensor, as an ambient sound-level sensor, as an ambient gas sensor, as an ambient voltage sensor, as an ambient humidity sensor, as an ambient acid sensor and/or ambient base sensor, or the like. The sensor unit 12 may comprise a multiplicity of sensor elements for sensing at least one operator-specific characteristic quantity and/or at least one environment-specific characteristic quantity, there being conceivable, in particular, a combination of a great variety of sensor elements that are designed to sense differing operator-specific characteristic quantities and/or differing environment-specific characteristic quantities.

The safety garment 10 additionally comprises at least one communication unit 14, by means of which the sensed operator-specific characteristic quantity and/or the sensed environment-specific characteristic quantity can be transmitted to at least the further safety garment 16 for the purpose of communicating with the further safety garment 16. For the purpose of communicating with the safety garment 10, the further safety garment 16 likewise comprises at least one communication unit 42. In addition the further safety garment 16 likewise comprises at least one sensor unit 44, for sensing at least one operator-specific characteristic quantity and/or at least one environment-specific characteristic quantity.

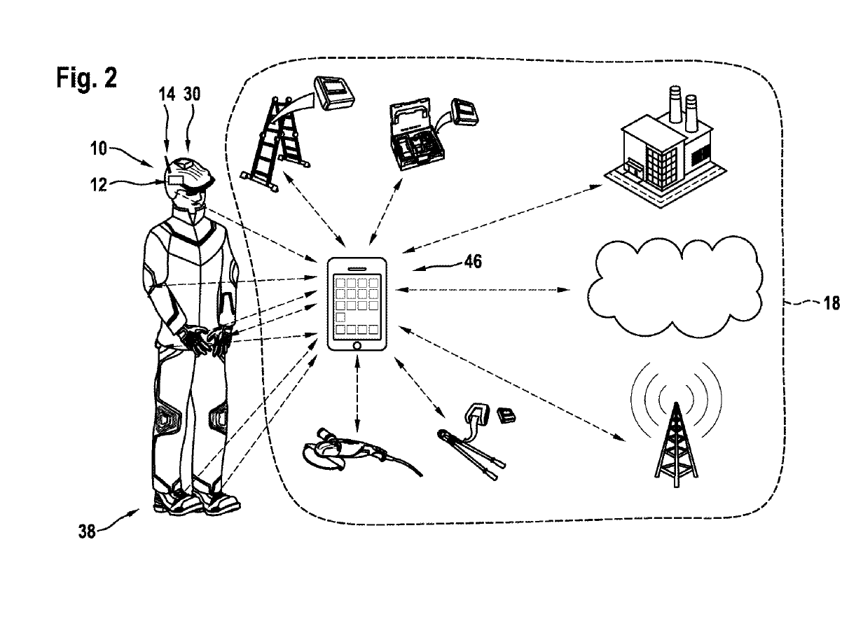

The communication unit 14 of the safety garment 10 is designed, moreover, to communicate with at least one external unit 18, realized so as to be different from a safety garment, for the purpose of exchanging electronic data (FIG. 2). The external unit 18 is realized as a hand-held power tool or as a company control center. It is also conceivable, however, that the external unit 18 is of a different design, considered appropriate by persons skilled in the art, such as, for example, designed as an external communication module that can be attached to various objects such as, for example, to a ladder, a tool, a toolbox, or the like, designed as a cloud, designed as a mobile telephony network, or the like. It is conceivable in this case for the communication unit 14 to communicate with the external unit 18 via a router unit 46 of the safety system 38, or directly. In the case of communication via an external router unit 46, the external router unit 46 is preferably realized as a portable communication device, in particular as a smartphone.

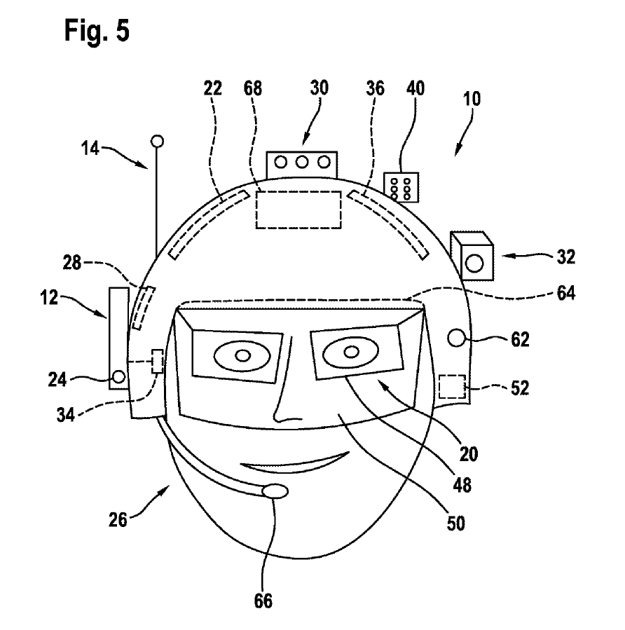

Furthermore, the safety garment 10 comprises at least one output unit 20, which is designed to output information in dependence on the sensed operator-specific characteristic quantity, the sensed environment-specific characteristic quantity, and/or in dependence on data received by means of the communication unit 14 of the safety garment 10 (FIG. 5). For this purpose, the output unit 20 of the safety garment 20 comprises at least one optical output element 48. The optical output element 48 is preferably realized as a display. If the safety garment 10 is realized as a safety helmet, the optical output element 48 is integrated into a visor element 50 of the safety garment 10 (FIG. 5). It is conceivable in this case that the optical output element 48 can be connected-in optionally, in particular only if required. The optical output element 48 is only visible in the visor element 50 if there is a requirement or if a wearer of the safety garment 10 activates the optical output element 48. It is also conceivable, however, that the optical output element 48 is realized so as to be separate from the visor element 50, and is disposed as a separate element on the safety garment 10, in particular disposed in a movable manner on the safety garment 10 and, in particular, is movable, when required, into a field of view of a wearer of the safety garment 10.

The output unit 20 of the safety garment 10 additionally comprises at least one acoustic output element 52. The acoustic output element 52 is realized as a loudspeaker. If the safety garment 10 is designed as a safety helmet, the acoustic output element 52 is integrated into an ear protection region of the safety helmet. It is also conceivable, however, that the optical output element 52 is realized so as to be separate from the safety garment 10, in particular from the safety helmet, and is realized, for example, as a headset that can be disposed on the safety garment 10, in particular on the safety helmet, in particular disposed in a movable manner on the safety helmet. Moreover, it is conceivable that the output unit 20 of the safety garment 10 has at least one further output element, which is realized as a tactile output element such as, for example, as a vibration generator. The output unit 20 of the safety garment 10 can be used to output, at least, safety instructions that are dependent, at least, on data received by means of the communication unit 14 of the safety garment 10. Moreover, items of information that are dependent, at least, on at least one characteristic quantity sensed by means of the sensor unit 12 of the safety garment 10 can be output by means of the output unit 20 of the safety garment 10. It is conceivable in this case that, for example a noise level can be sensed by means of a sensor element, in particular a noise sensor element, of the sensor unit 12. This noise level can be evaluated, for example, by means of an evaluation unit 22 of the safety garment 10 (FIG. 5). If it can be ascertained that a limit value of a noise level is exceeded, for example a corresponding item of information, in particular a warning signal and/or a warning symbol, can be output by means of the output unit 20 of the safety garment 10. In addition, it is conceivable that further characteristic quantities, considered appropriate by persons skilled in the art, can be sensed by means of the sensor unit 12 of the safety garment 10, which characteristic quantities can be evaluated by means of the evaluation unit 22 of the safety garment 10, and corresponding items of information can be output by means of the output unit 20 of the safety garment 10. In this case, for example, an ambient brightness, a status of a state of the safety garment 10, the presence of radioactivity, an acid, a gas, a dust burden, etc., an ambient temperature, a base surface state, application of a voltage, overloading of a wearer of the safety garment 10, such as, for example, as a result of incorrect posture, as a result of lifting an excessively heavy load, or the like, a number of steps, a weight of a wearer of the safety garment 10, a daily output of a wearer of the safety garment 10, an access authorization to a region and/or a space of a work area, calories used, or the like, can be sensed by means of the sensor unit 12 of the safety garment 10. The sensed characteristic quantities can be evaluated by means of the evaluation unit 22 of the safety garment 10, and corresponding items of information can be output by means of the output unit 20 of the safety garment 10.

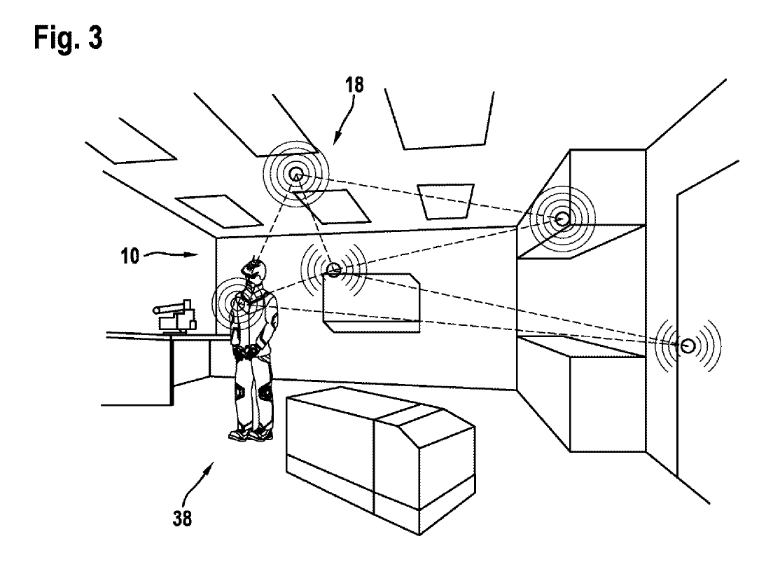

The at least one external unit 18 has at least one access control function, which is designed to monitor, enable or block access of a wearer of the safety garment 10 and/or of the further safety garment 16 to a work area or a space (FIG. 3). Further data, which can be evaluated by means of the communication unit 14 of the safety garment 10 and/or the communication unit 42 of the further safety garment 16 can be monitored and/or recorded by means of the external unit 18. The sensor unit 12 of the safety garment 10 has at least one eye sensor element 34, which is designed to sense at least one eye characteristic quantity of a wearer of the safety garment 10, the eye characteristic quantity being able to be transmitted to the further safety garment 16 and/or to the external unit 18 by means of the communication unit 14 (FIG. 5). This renders possible, advantageously, a viewing direction of a wearer of the safety garment 10, such as, for example, for outputting work instructions, and/or monitoring of fatigue of a wearer of the safety garment 10.

The safety garment 10 comprises at least the evaluation unit 22, which is designed to evaluate a safety garment combination, at least in dependence on a communication with the further safety garment 16. When the safety garment 10 and the further safety garment 16 are being worn, a communication is effected by means of the communication unit 14 of the safety garment 10 and the communication unit 42 of the further safety garment 16, the safety garment 10 and the further safety garment 16 connecting mutually to each other and transmitting this to the external unit 18. The at least one external unit 18 has at least one garment monitoring function, which is designed to monitor a safety garment combination that is contingent upon a work region and/or a work assignment, in dependence on safety regulations and/or operating-area regulations. A correct safety garment combination is preferably pre-evaluated by means of the evaluation unit 22 of the safety garment 10, the pre-evaluation being at least partially dependent on safety regulations and/or operating-area regulations transmitted by means of the communication unit 14 of the safety garment 10.

Moreover, the evaluation unit 22 of the safety garment 10 is designed to evaluate a sensed environment, at least in dependence on an environment-sensing sensor element 24, in particular a camera, of the sensor unit 12 of the safety garment 10, in order to make possible a work instruction. For example, it is possible to sense a workpiece on which work is to be performed and which can be evaluated, by means of the evaluation unit 22 of the safety garment 10, for example in dependence on an electronic work plan that is present. For example, it can be ascertained at what location work is to be performed on the workpiece in order to fulfill the work plan. This can be output to a wearer of the safety garment 10 by means of the output unit 20 of the safety garment 10, the workpiece itself being able to be used, in particular, as a part of an output of a work instruction; for example, in the case of a view directed toward the workpiece, a corresponding work instruction can be output in dependence on a position of the workpiece relative to the safety garment 10. It is also conceivable, however, that the workpiece can only be represented digitally by means of the output unit 20 of the safety garment 10, and that work instructions can accordingly be represented digitally. The safety system 38 and/or at least the safety garment 10 are/is designed for virtual-reality working and/or augmented-reality working of a workpiece.

Furthermore, the safety garment 10 has at least one input unit 26, at least for inputting operator-specific control commands, wherein, for the purpose of controlling the external unit 18, in particular a hand-held power tool, the operator-specific control commands can be transmitted to the external unit 18 by means of the communication unit 14. The input unit 26 of the safety garment 10 comprises at least one haptic input element 62, for inputting operator-specific control commands. The haptic input element 62 may be realized as a key, as a switch, as a contact-sensitive display, or as another input element 62 considered appropriate by persons skilled in the art. The input unit 26 of the safety garment 10 has at least one gesture control function and/or voice-control function. The input unit 26 of the safety garment 10 comprises at least one gesture-control input element and/or at least one voice-control input element 66. The gesture-control input element 64 is preferably realized as a camera. Sensed gestures of a wearer of the safety garment 10 can preferably be evaluated by means of the evaluation unit 22 of the safety garment 10, and processed by means of an open-loop and/or closed-loop control unit 68 of the safety garment 10 to create control commands. The voice-control input element 66 is preferably realized as a microphone. Sensed voice commands of a wearer of the safety garment 10 can preferably be evaluated by means of the evaluation unit 22 of the safety garment 10, and processed by means of the open-loop and/or closed-loop control unit 68 of the safety garment 10 to create control commands.

The safety garment 10 additionally comprises at least one actuator unit 28, which can be controlled at least in dependence on the sensed operator-specific characteristic quantity, in dependence on the sensed environment-specific characteristic quantity and/or in dependence on data received by means of the communication unit 14 of the safety garment 10 (FIG. 5). The actuator unit 28 of the safety garment 10 is designed, at least, to act actively upon a wearer as a result of identification of a hazard situation. The further safety garment 16 likewise comprises at least one actuator unit 70, which can be controlled at least in dependence on the sensed operator-specific characteristic quantity, in dependence on the sensed environment-specific characteristic quantity and/or in dependence on data received by means of the communication unit 42 of the further safety garment 16. The actuator unit 70 of the further safety garment 16 is designed, at least, to act actively upon a wearer as a result of identification of a hazard situation. If the further safety garment 16 is realized as a work jacket, the actuator unit 70 of the further safety garment 16 is disposed, for example, in a sleeve of the further safety garment 16 realized as a work jacket. The actuator unit 70 of the further safety garment 16 may be integrated directly into the material, such as, for example, as a smart material, or the actuator unit 70 of the further safety garment 16 may be disposed in a removable manner on the further safety garment 16. Upon identification of a hazard situation, an active action of the actuator unit 28 of the safety garment 10 and/or an action of the actuator unit 70 of the further safety garment 16 can cause a movement by a wearer of the safety garment 10 and/or of the further safety garment 16, which movement enables the wearer of the safety garment 10 and/or of the further safety garment 16 to avert the hazard situation, in particular enables the wearer of the safety garment 10 and/or of the further safety garment 16 to move out of the hazard situation. The sensor unit 12 of the safety garment 10 has at least one position sensor element 40, the external unit 18 having at least one hazard identification function, which is designed to evaluate, at least, a hazard situation of a wearer of the safety garment, at least in dependence on a position characteristic quantity sensed by means of the position sensor element 40.

Furthermore, the safety garment 10 comprises at least one lighting unit 30 for illuminating a work area, wherein the lighting unit 30 can be controlled at least in dependence on the sensed operator-specific characteristic quantity, in dependence on the sensed environment-specific characteristic quantity and/or in dependence on data received by means of the communication unit 14 of the safety garment 10 (FIG. 5). The safety garment 10 additionally comprises at least one projection unit 32, which is designed to project at least one item of information onto a base, wherein the projection unit 32 of the safety garment 10 can be controlled at least in dependence on the sensed operator-specific characteristic quantity, in dependence on the sensed environment-specific characteristic quantity and/or in dependence on data received by means of the communication unit 14 of the safety garment 10 (FIG. 5).

The safety garment 10 additionally comprises at least one energy supply unit 36, which is designed to supply at least the sensor unit 12 of the safety garment 10 and the communication unit 14 with energy in dependence on a handling of an external unit 18, in particular a hand-held power tool. If the external unit 18 is realized as a hand-held power tool, it is conceivable that the energy supply unit 36 has at least one connecting element (not represented in greater detail here), which is designed to be connected to the hand-held power tool, in particular to an energy supply unit and/or to an energy storage unit of the hand-held power tool. The connecting element in this case may be of a wired design such as, for example, as a power supply cable or the like, or of a wireless design such as, for example, as a primary coil, as an NFC or the like.

The safety system 38 comprises, in addition to the safety garment 10 and the further safety garment 16, at least one additional safety garment realized as work trousers 54, an additional safety garment realized as a safety glove 56, an additional safety garment realized as a safety shoe 58 and/or an additional safety garment realized as safety glasses 60 (FIG. 1). Preferably, all safety garments of the safety system 38 each have at least one communication unit for communicating with each other, and/or at least one sensor unit for sensing at least one operator-specific characteristic quantity and/or at least one environment-specific characteristic quantity. Preferably, all safety garments of the safety system 38 can exchange electronic data with each other, conceivable data being, for example, data for evaluating, warning, controlling and/or for use of other functions. Preferably, all safety garments of the safety system 38 have all units that the safety garment 10 realized as a safety helmet also has. A method for operating the safety system 38 comprises an at least partially automatic communication of the individual safety garments with each other and/or with the external unit 18. In a further method step, advantageously, functions of the safety system 38 are controlled at least partially automatically, in particular in dependence on sensed characteristic quantities. In respect of further method steps of the method for operating the safety system 38, reference may be made to the description of the safety system 38 and/or of the safety garment 10, since this description is to be construed as applying analogously to the method, and therefore all features in respect of the safety system 38 and/or of the safety garment 10 are also deemed as disclosed with regard to the method for operating the safety system 38.

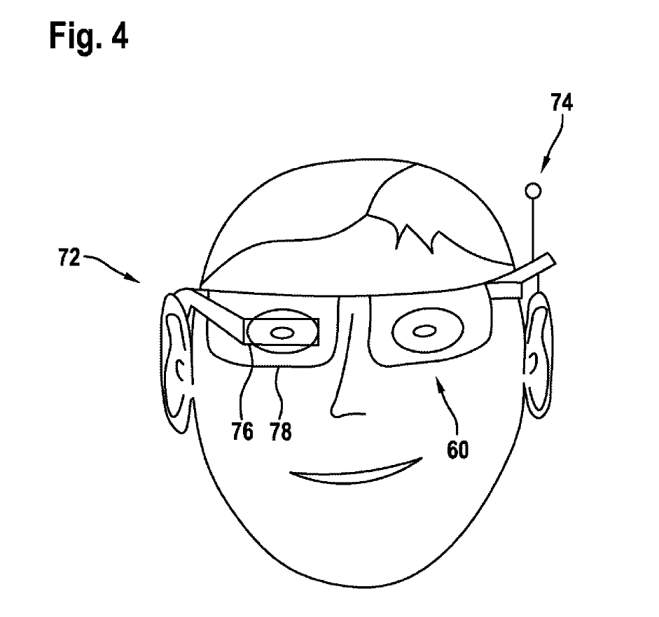

FIG. 4 shows a detail view of the additional safety garment of the safety system 38 that is realized as safety glasses 60. Preferably, the additional safety garment realized as safety glasses 60 has all units that the safety garment 10 realized as a safety helmet also has. The additional safety garment realized as safety glasses 60 comprises at least one output unit 72, which is designed to output items of information in dependence on at least one sensed position characteristic quantity, at least one sensed environment-specific characteristic quantity and/or in dependence on data received by means of a communication unit 74 of the additional safety garment realized as safety glasses 60. For this purpose, the output unit 72 of the additional safety garment realized as safety glasses 60 comprises at least one optical output element 76. The optical output element 76 of the additional safety garment realized as safety glasses 60 is preferably realized as a display. The optical output element 76 of the additional safety garment realized as safety glasses 60 is integrated into at least one lens element 78 of the additional safety garment realized as safety glasses 60. Moreover, it is conceivable that the optical output element 76 of the additional safety garment realized as safety glasses 60 can be connected-in optionally, in particular only if required. The optical output element 76 of the additional safety garment realized as safety glasses 60 is only visible in the lens element 78 if there is a requirement or if a wearer of the additional safety garment realized as safety glasses 60 activates the optical output element of the additional safety garment realized as safety glasses 60. It is also conceivable, however, that the optical output element 76 of the additional safety garment realized as safety glasses 60 is realized so as to be separate from the lens element 78, and is disposed as a separate element on the additional safety garment realized as safety glasses 60, in particular disposed in a movable manner on the additional safety garment realized as safety glasses 60 and, in particular, is movable, when required, into a field of view of a wearer of the additional safety garment realized as safety glasses 60.

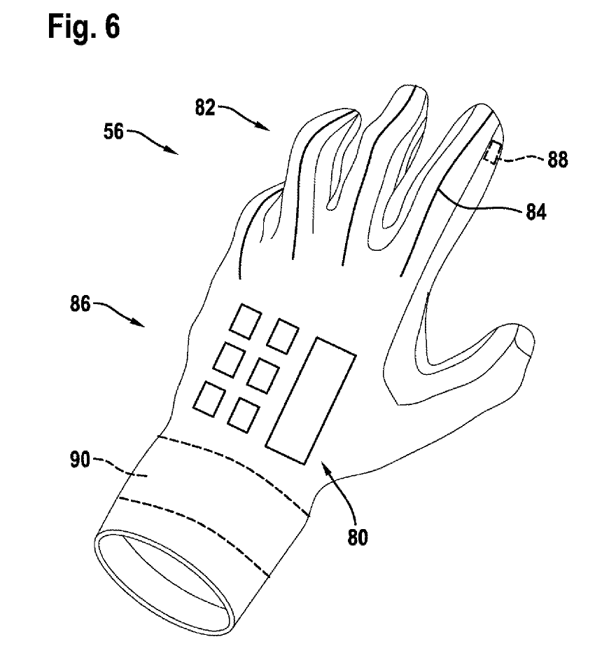

FIG. 6 shows a detail view of the additional safety garment of the safety system 38 realized as a safety glove 56. Preferably, the additional safety garment realized as a safety glove 56 has all units that the safety garment 10 realized as a safety helmet also has. The additional safety garment realized as a safety glove 56 has at least one output unit 80 and a lighting unit 82, which are realized so as to be at least partially integral. It is conceivable in this case for incorporated fiber-optic elements 84, in particular glass fibers, to be provided to enable an optical feedback such as, for example, concerning a state of the safety glove 56, a warning indication, an operating mode/state of the external unit 18. The fiber-optic elements 84 may extend, for example, on a region of the safety glove 56 that covers the back of the hand, over individual finger regions of the safety glove 56, to finger-tip regions of the safety glove 56. At the finger-tip regions of the safety glove 56, the fiber-optic elements 84 may be provided as lighting elements for illumination. Moreover, it is conceivable that the output unit 80 of the additional safety garment realized as a safety glove 56 comprises vibration elements that are disposed, for example, in the finger-tip regions of the safety glove 56. This makes it possible, advantageously, to achieve an additional feedback capability.

The additional safety garment realized as a safety glove 56 additionally comprises at least one input unit 86. The input unit 86 of the additional safety garment realized as a safety glove 56 comprises, for example, input elements 88, in particular pressure-sensitive sensors, that are disposed in the finger-tip regions of the safety glove 56. It is conceivable in this case that, for example, particular movement patterns or particular pressure patterns can be interpreted as commands. In addition, movements of individual finger-tip regions of the safety glove 56 can be interpreted, for example, for the purpose of gesture control by means of the gesture control function of the safety garment 10 realized as a safety helmet. Further, it is conceivable that, by means of the safety glove 56, in particular in combination with the sensor unit 12 of the safety garment 10 realized as a safety helmet, measurement of distances can be achieved, by displaying distances by the spreading of fingers, in particular of a thumb and an index finger, a measurement value being able to be output by means of an output unit of the safety system 38. It is additionally conceivable that the additional safety garment realized as a safety glove 56 has a wall scanner function, elements detected in a wall being able to be output by means of an output unit of the safety system 38. Other functions of individual safety garments of the safety system 38 and/or of the safety system 38 itself, considered appropriate by persons skilled in the art, are likewise conceivable.

Furthermore, the additional safety garment realized as a safety glove 56 comprises at least one energy supply unit 90, which is designed to supply an electrical energy supply with energy in dependence on a handling of the external unit 18, in particular a hand-held power tool. A contactless, in particular inductive, transfer of energy is made possible as a result the external unit 18 being gripped.

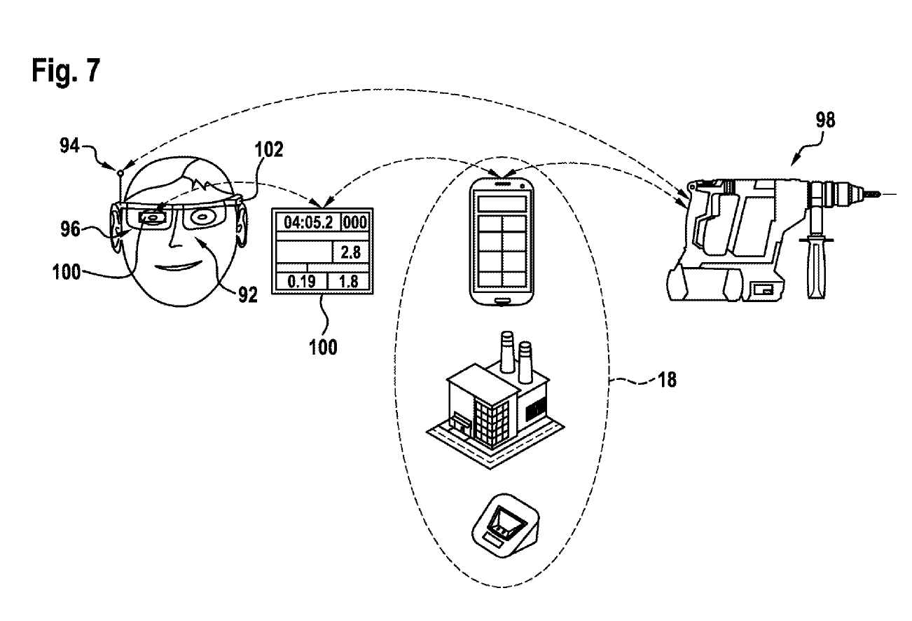

FIG. 7 shows a schematic representation of a use of a safety garment realized as an eye protection device 92 in the safety system 38. The eye protection device 92 is realized as safety glasses. It is also conceivable, however, that the eye protection device 92 is realized as a safety visor that is movably mounted on a safety helmet, or that can be disposed separately on the head of an operator, in particular by means of a retaining band or the like. The eye protection device 92 is designed for use in the safety system 38. It is also conceivable, however, that the eye protection device 92 can be used independently of the safety system 38. The eye protection device 92 comprises at least one communication unit 94 for exchanging electronic data. The communication unit 94 is designed to communicate with at least one hand-held power tool 98. The communication unit 94 is designed, in particular additionally, at least to communicate with an external unit 18 that is different from the hand-held power tool 98. The communication unit 94 is designed, at least, to communicate with an external unit 18 realized as a mobile device, in order to exchange electronic data with the mobile device. In particular, the mobile device is realized as a smartphone. Advantageously, it is possible to achieve outputting of incoming short message calls such as, for example, an SMS, or the like.

The communication unit 94 is realized, at least, to communicate with an external unit 18 realized as a charging device, in order to receive at least one charge characteristic quantity from at least one storage battery that can be charged by means of the charging device. The charge characteristic quantity may be realized as a charge state, as a remaining charge duration, as a charge capacity, as a charging voltage, or the like.

The eye protection device 92 has at least one output unit 96, which is designed to output items of information in dependence on data received by means of the communication unit 94. The output unit 96 may be realized as a haptic, acoustic and/or optical output unit. The output unit 96 can preferably be controlled by open-loop and/or closed-loop control, by means of an open-loop and/or closed-loop control unit (not represented in greater detail here) of the eye protection device 92. The output unit 96 is preferably designed to output work instructions, in particular optical and/or acoustic work instructions, that are dependent, at least, on data received by means of the communication unit 94.

Preferably, the output unit 96 comprises at least one output element 100, which is disposed on the eye protection device 92, in particular on a frame unit or on a protective lens unit of the eye protection device 92. In FIG. 7, to aid illustration, the output element 100 is additionally represented separately from the eye protection device 92. The output element 100 is designed to output at least one item of information. The output element 100 may be integrated into a lens, movably mounted on the frame unit, or the like. It is additionally conceivable that the output element 100 can be connected-in optionally, in particular only if required. The output element 100 is only visible, for example, in the lens of the eye protection device 92 if there is a requirement or if a wearer of the eye protection device 92 activates the output element 100. It is also conceivable, however, that the output element 100 of the eye protection device 92 is realized so as to be separate from the lens of the eye protection device 92, and is disposed as a separate element on the eye protection device 92, in particular disposed in a movable manner on the eye protection device 92 and, in particular, is movable, when required, into a field of view of a wearer of the eye protection device 92. The output element 100 is realized as an optical output element. The output element 100 is preferably realized as a display. It is also conceivable, however, that the output element 100, alternatively or additionally, is realized as an acoustic output element.

The eye protection device 92 may have a sensor unit 102 in addition to the communication unit 94. The sensor unit 102 may be of a design that is at least substantially similar to that of the sensor unit 12 of the safety garment 10. The sensor unit 102 of the eye protection device 92 has at least one eye sensor element (not represented in greater detail here), which is designed to sense at least one eye characteristic quantity of a wearer of the eye protection device 92, the eye characteristic quantity being able to be processed by the open-loop and/or closed-loop control unit of the eye protection device 92 and/or being able to be transmitted to the hand-held power tool 98 and/or to the external unit 18 by means of the communication unit 94. Preferably the eye sensor element is designed, at least, to sense an eye characteristic quantity realized as a state (open, closed, blink rate, or the like) of at least one eye of a wearer of the eye protection device 92, or an eye characteristic quantity realized as a viewing direction of an eye of a wearer of the eye protection device 92. It is also conceivable, however, that the eye sensor element is designed, additionally or alternatively, to sense different or additional eye characteristic quantities, considered appropriate by persons skilled in the art. It is thus possible, advantageously, to deduce the attentiveness of a wearer of the eye protection device 92, since it is possible to identify, for example, that the wearer has directed their view away from a workpiece on which work is to be performed and/or from the hand-held power tool 98. It is conceivable that a safety switch-off of the hand-held power tool 98 can be initiated in dependence on the sensed eye characteristic quantity. Moreover, advantageously, identification of wearer fatigue can be achieved in dependence on the sensed eye characteristic quantity. Advantageously, a warning signal can be output.

For the purpose of supplying energy to differing units of the eye protection device 92, the eye protection device 92 comprises, in particular, an energy supply unit (not represented in greater detail here). The energy supply unit of the eye protection device 92 is preferably realized as a storage battery. The eye protection device 92 may have a contactless and/or wired charging connection unit for recharging the energy supply unit.

In addition to the already described units of the eye protection device 92, the eye protection device 92 may additionally have further units, such as have already been described in connection with the safety garments mentioned in this description.

1. A safety garment comprising:
at least one sensor unit configured to sense at least one operator-specific characteristic quantity and/or at least one environment-specific characteristic quantity; and
at least one communication unit configured to transmit the sensed operator-specific characteristic quantity and/or the sensed environment-specific characteristic quantity to at least one further safety garment to communicate with the further safety garment.

2. The safety garment as claimed in claim 1, wherein:
the communication unit is configured to exchange electronic data with at least one external unit, and
the external unit is different from a safety garment.

3. The safety garment as claimed in claim 1, further comprising:
at least one output unit configured to output items of information in dependence on the sensed operator-specific characteristic quantity, the sensed environment-specific characteristic quantity and/or in dependence on data received by the communication unit.

4. The safety garment as claimed in claim 3, wherein the output unit is configured to output at least work instructions that are dependent, at least, on the data received by the communication unit.

5. The safety garment as claimed in claim 1, further comprising:
at least one evaluation unit configured to evaluate a safety garment combination, at least in dependence on the communication with the further safety garment.

6. The safety garment as claimed in claim 1, further comprising:
at least one evaluation unit configured to evaluate a sensed environment, at least in dependence on an environment-sensing sensor element of the sensor unit, in order to make possible a work instruction.

7. The safety garment as claimed in claim 1, further comprising:
at least one input unit configured to input operator-specific control commands,
wherein the communication unit transmits the operator-specific control commands to an external unit to control the external unit.

8. The safety garment as claimed in claim 7, wherein the input unit has at least one gesture control function and/or voice control function.

9. The safety garment as claimed in claim 1, further comprising:
at least one actuator unit configured for control at least in dependence on the sensed operator-specific characteristic quantity, in dependence on the sensed environment-specific characteristic quantity, and/or in dependence on data received by the communication unit.

10. The safety garment as claimed in claim 9, wherein the actuator unit is configured to act actively on a wearer as a result of identification of a hazard situation.

11. The safety garment as claimed in claim 1, further comprising:
at least one lighting unit configured to illuminate a work area,
wherein the lighting unit is controllable at least in dependence on the sensed operator-specific characteristic quantity, on the sensed environment-specific characteristic quantity, and/or on the data received by the communication unit.

12. The safety garment as claimed in claim 1, further comprising:
at least one projection unit configured to project at least one item of information onto a base,
wherein the projection unit is configured for control at least in dependence on the sensed operator-specific characteristic quantity, on the sensed environment-specific characteristic quantity, and/or on data received by the communication unit.

13. The safety garment as claimed in claim 1, wherein:
the sensor unit has at least one eye sensor element configured to sense at least one eye characteristic quantity of a wearer, and
the eye characteristic quantity is transmitted to the further safety garment and/or to an external unit by the communication unit.

14. The safety garment as claimed in claim 1, further comprising:
at least one energy supply unit configured to supply at least the sensor unit and the communication unit with energy in dependence on a handling of an external unit.

15. A safety system, comprising:
at least one safety garment including (i) at least one sensor unit configured to sense at least one operator-specific characteristic quantity and/or at least one environment-specific characteristic quantity, and (ii) at least one communication unit configured to transmit the sensed operator-specific characteristic quantity and/or the sensed environment-specific characteristic quantity to at least one further safety garment to communicate with the further safety garment; and
at least one further safety garment or at least one external unit,
wherein the at least one safety garment is configured to exchange electronic data with the further safety garment or the at least one external unit with the communication unit.

16. The safety system as claimed in claim 15, wherein:
the sensor unit of the safety garment has at least one position sensor element, and
the external unit has at least one hazard identification function configured at least to evaluate a hazard situation of a wearer of the safety garment, at least in dependence on a position characteristic quantity sensed by the position sensor element.

17. The safety system as claimed in claim 15, wherein the at least one external unit has at least one access control function configured to monitor, enable, or block access of a wearer of the safety garment and/or of the further safety garment to a work area or a space.

18. The safety system as claimed in claim 15, wherein the at least one external unit has at least one garment monitoring function configured to monitor a safety garment combination that is contingent upon a work region and/or a work assignment, in dependence on safety regulations and/or operating-area regulations.

19. The safety system as claimed in claim 15, wherein the at least one external unit is a hand-held power tool or a company control center.

20. The safety system as claimed in claim 15 further comprising:
an eye protection device having (i) at least one further communication unit configured to exchange electronic data, the further communication unit configured to communicate with at least one hand-held power tool, and (ii) at least one output unit configured to output items of information in dependence on data received by the further communication unit,
wherein the further communication unit is configured to communicate with a further external unit that is different from a hand-held power tool.

21. The safety system as claimed in claim 20, wherein the further communication unit is configured to communicate with the further external unit realized as a mobile device, in order to exchange data with the mobile device.

22. The safety system as claimed in claim 20, wherein the further communication unit is configured to communicate with the further external unit realized as a charging device, in order to receive at least one charge characteristic quantity from at least one storage battery that is chargeable by the charging device.

23. The safety system as claimed in claim 20, wherein the further communication unit is configured to communicate with the further external unit realized as a company control center, in order to receive at least project data.

24. (canceled)
